(19) United States
(12) Patent Application Publication
(10) Pub. No.: US 2021/0292817 A1
(43) Pub. Date: Sep. 23, 2021

(54) METHODS FOR QUANTITATION OF ANALYTES IN MULTIPLEXED BIOCHEMICAL REACTIONS

(71) Applicant: ChromaCode, Inc., Carlsbad, CA (US)

(72) Inventors: Aditya RAJAGOPAL, Carlsbad, CA (US); Christopher MACDONALD, Carlsbad, CA (US)

(21) Appl. No.: 17/258,880

(22) PCT Filed: Jul. 10, 2019
(86) PCT No.: PCT/US2019/041239

§ 371 Date: Jan. 8, 2021

Related U.S. Application Data
(60) Provisional application No. 62/696,558, filed on Jul. 11, 2018.

(51) Int. Cl. C12Q 1/6816; G16B 40/00; G01N 21/64; G01N 21/76

(52) U.S. Cl. CPC C12Q 1/6816; G16B 40/00; G01N 2021/6439; G01N 21/76; G01N 21/6428

(57) ABSTRACT

The present disclosure provides methods, systems, and compositions of quantifying one or more analytes in a single sample volume without immobilization, separation, mass spectrometry, or melting curve analysis. Methods may comprise polymerase chain reaction and signal generation to quantify the analytes.

### CROSS-REFERENCE

This application claims the benefit of U.S. Provisional Patent Application No. 62/696,558, filed Jul. 11, 2018, and is incorporated by reference in its entirety for all purposes.

### BACKGROUND

In PCR, detection of multiple target nucleic acid sequences in a single reaction is accomplished by associating each nucleic acid target with a distinct fluorescent tag. PCR may be used to amplify nucleic acids for analysis. The present disclosure provides a precise method of quantifying the presence of one or more analytes in a single sample volume without immobilization, separation, mass spectrometry, or melting curve analysis.

### SUMMARY

Disclosed herein, in some aspects, are methods, systems and compositions for quantifying nucleic acids in a sample. In an aspect, the present disclosure provides a method of quantifying at least a first and a second nucleic acid in a sample, the method comprising: (a) providing a mixture comprising: (i) the first nucleic acid and the second nucleic acid; (ii) a first detection probe configured to generate a first signal when in the presence of the first nucleic acid and when subjected to reaction conditions; (iii) a second detection probe configured to generate a second signal when in the presence of the second nucleic acid and when subjected to reaction conditions; (b) subjecting the mixture to the reaction conditions thereby generating the first and second signal; (c) measuring (i) an intensity of the first signal in first range of wavelengths, (ii) an intensity of the first signal in the second range of wavelengths, (iii) an intensity of the second signal in a third range of wavelengths; (d) generating a first data set derived from the intensity (i) and the intensity (ii) measured in c) and generating a second data set derived from the intensity (iii) measured in (c); and (e) processing the generated first data set and the generated second data set, wherein the processing uses reference quantification parameters derived from a reference data set(s) to generate quantification parameters of the generated first and second data set, wherein the reference data set(s) corresponds to a reference condition(s), wherein the reference condition(s) comprises a quantity of reference nucleic acid(s), thereby quantifying the first and second nucleic acid.

In some embodiments, the method further comprises in (c) measuring an intensity of the first or second signal is (iii) a third range of wavelengths. In some embodiments, the measuring comprises detecting the first or second signal using a multi-channel detector. In some embodiments, the first or second signal comprises electromagnetic radiation. In some embodiments, the first or second signal is generated by fluorescence emission. In some embodiments, the first or second signal is generated by chemiluminescence. In some embodiments, the first range of wavelengths and the third range of wavelengths comprise a same wavelength. In some embodiments, the first range of wavelengths and the third range of wavelengths do not comprise a same wavelength.

In some embodiments, the processing comprises fitting the first or second data set to a curve. In some embodiments, the first or second data set is plotted as a curve. In some embodiments, the first or second data set is a kinetic signature. In some embodiments, the first or second nucleic acid comprises DNA. In some embodiments, the first or second nucleic acid comprises RNA.

In some embodiments, the method further comprises in (c), measuring an intensity of the second signal in (iv) a fourth range of wavelengths, and further comprising in d) generating the second data derived from the intensity iii) and the intensity of the second signal in the fourth range of wavelengths. In some embodiments, the fourth range of wavelength is the same as the first range of wavelength or the second range of wavelengths. In some embodiments, the third range of wavelengths is the same as the first range of wavelength or the second range of wavelengths. In some embodiments, the processing comprises identifying data points as corresponding to the first data set. In some embodiments, the processing comprises identifying data points as corresponding to the second data set. In some embodiments, the quantifying comprises calculating a relative quantification. In some embodiments, the relative quantification is generated by comparing the first data set and the second data set.

In another aspect, the present disclosure provides a system comprising a controller comprising or capable of accessing, computer readable media comprising non-transitory computer-executable instructions which, when executed by at least one electronic processor perform a method comprising: (a) providing a mixture comprising: (i) the at least one nucleic acid; (ii) at least a first detection probe configured to generate a signal when in the presence of the at least one nucleic acid and when subjected to reaction conditions; (b) subjecting the mixture to the reaction conditions, thereby generating the signal; (c) measuring (i) an intensity of the signal in a first range of wavelengths and (ii) an intensity of the signal in a second range of wavelengths; (d) generating a data set derived from the intensities measured in (c); and (e) processing the generated data set, wherein the processing uses reference quantification parameters derived from a reference data set(s) to calculate quantification parameters of the generated data set, wherein the reference data set(s) corresponds to a reference condition(s), wherein the reference condition(s) comprises a quantity of reference nucleic acid, thereby quantifying the at least one nucleic acid.

In another aspect, the present disclosure provides a system for the quantification of at least one nucleic acid in a sample comprising: (a) the sample comprising the at least one nucleic acid; (b) a first detection probe configured to generate a signal when in the presence of the at least one nucleic acid and when subjected to reaction conditions; (c) a detector or plurality of detectors configured to measure (i) an intensity of the signal in a first range of wavelengths and (ii) an intensity of the signal in a second range of wavelengths; and (d) a processor configured to: (i) generate a data set derived from the measured intensities of (c); and (ii) process the generated data set by using reference quantification parameters derived from a reference data set(s) to calculate quantification parameters of the generated data set, wherein the reference data set(s) corresponds to a reference condition(s), wherein the reference condition(s) comprises a quantity of reference nucleic acid.

In some embodiments, the system further comprises in (c) detectors configured to measure (iii) an intensity of the signal is a third range of wavelengths. In some embodiments, the detector comprises a multi-channel detector. In some embodiments, the quantification comprises calculating an absolute quantification.

In another aspect, the present disclosure provides a method of quantifying at least one nucleic acid in a sample volume, the method comprising: (a) providing a mixture comprising: (i) the at least one nucleic acid; (ii) a first detection probe configured to generate a signal when in the presence of the at least one nucleic acid and when subjected to reaction conditions; (b) subjecting the mixture to the reaction conditions, thereby generating the signal; (c) measuring (i) an intensity of the signal in a first range of wavelengths and (ii) an intensity of the signal in a second range of wavelengths; (d) generating a data set derived from the intensities measured in c); and (e) processing the generated data set, wherein the processing uses reference quantification parameters derived from a reference data set(s) to calculate quantification parameters of the generated data set, wherein the reference data set(s) corresponds to a reference condition(s), wherein the reference condition(s) comprises a quantity of reference nucleic acid(s), thereby quantifying the at least one nucleic acid.

In some embodiments, the method does not comprise immobilization, separation, mass spectrometry, or melting curve analysis. In some embodiments, the method further comprise in (c) measuring (iii) an intensity of the signal in a third range of wavelengths.

In some embodiments, subjecting the mixture the reaction conditions comprises applying electromagnetic radiation to the mixture. In some embodiments, the measuring comprises detecting the signal using a multi-channel detector. In some embodiments, the signal comprises electromagnetic radiation. In some embodiments the electromagnetic radiation comprises a wavelength of electromagnetic radiation. In some embodiments, the wherein the electromagnetic radiation comprises a plurality of wavelengths of electromagnetic radiation. In some embodiments, the signal is generated by fluorescence emission. In some embodiments, the signal is generated by chemiluminescence. In some embodiments, the first range of wavelengths and the second range of wavelengths comprise a same wavelength. In some embodiments, the first range of wavelengths and the second range of wavelengths do not comprise a same wavelength.

In some embodiments, the detection probe is configured to anneal to the at least one nucleic acid. In some embodiments, the detection probe is configured to generate the signal upon degradation of a portion of the detection probe. In some embodiments, detection probe comprises a fluorophore or dye. In some embodiments, the mixture further comprises an amplification oligomer. In some embodiment, the amplification oligomer comprises a sequence complementary to a portion of the sequence of the at least the nucleic acid. In some embodiments, the reaction conditions comprise conditions for a DNA extension reaction. In some embodiments, the DNA extension reaction is a polymerase chain reaction (PCR). In some embodiments, the polymerase chain reaction is a quantitative polymerase chain reaction (qPCR).

In some embodiments, the processing comprises using a mathematical algorithm. In some embodiments, the mathematical algorithm comprises expectation maximization, nearest neighbor analysis, basic model parameterization or Bayesian estimation. In some embodiments, the the mathematical algorithm comprises a process parameter. In some embodiments, the process parameter comprises a) a threshold cycle, b) an amplitude, or c) a slope. In some embodiments, the processing comprises fitting the generated data set to a curve. In some embodiments, the data set is plotted as a curve. In some embodiments, data set comprises a kinetic signature. In some embodiments, the reference condition(s) comprises an amplification reaction condition. In some embodiments, the reference conditions comprise a) a temperature, b) a pH, c) a concentration of the reference nucleic acid, or a combination thereof. In some embodiments, the reference data set corresponds with a data set generated by amplification of the reference nucleic acid under amplification parameters, wherein the reference data set is indicative of an amplification parameter comprising: a) a primer concentration, b) a polymerase concentration, c) polymerase type, d) a reference nucleic acid concentration, e) a number of thermocycles, f) a rate of thermocycling, g) a thermocycle time length, h) a probe sequence; i) a primer sequence, or combinations thereof.

In some embodiments, the quantifying comprises calculating an absolute quantification. In some embodiments, the reference data set is generated using predetermined concentrations of the reference nucleic acid.

In some embodiments, at least one nucleic acid is derived from a biological sample. In some embodiments, the biological sample is blood or plasma. In some embodiments, the biological sample is derived from a virus. In some embodiments, the at least one nucleic acid comprises DNA. In some embodiments, the DNA comprises genomic DNA. In some embodiments, the at least one nucleic acid comprises RNA. In some embodiments, the RNA comprises mRNA.

### BRIEF DESCRIPTION OF THE DRAWINGS

FIG. 1B illustrates an amplification curve resulting from a multiplex assay comprising two targets with similar, albeit distinct Cₜ points in a fluorescently multiplexed PCR. FIG. 1A illustrates a multiplex assay of two targets wherein one target is encoded with a fluorescent probe that fluoresces in more than one fluorescent channels, thereby generating two unique curve signatures for enhanced analytical study.

FIGS. 2A-2F illustrates a multiplex assay comprising different combinations of targets (A) Metapneumovirus and FluB (B) Metapneumovirus and Adenovirus (C) PIV1 and PIV3 (D) PIV 1 and PIV2 (E) PIV3 and PIV2 (F) RSVA and RSVB.

### DETAILED DESCRIPTION

The following description provides specific details for a comprehensive understanding of, and enabling description for, various embodiments of the technology. It is intended that the terminology used be interpreted in its broadest reasonable manner, even where it is being used in conjunction with a detailed description of certain embodiments.

Before describing the present teachings in detail, it is to be understood that the disclosure is not limited to specific compositions or process steps, and as such, may vary. As used herein, the singular forms “a,” “an,” and “the” are intended to include the plural forms as well, unless the context clearly indicates otherwise. Furthermore, to the extent that the terms “including,” “includes,” “having,” “has,” “with,” “such as,” or variants thereof, are used in either the specification and/or the claims, such terms are not limiting and are intended to be inclusive in a manner similar to the term “comprising.” Unless specifically noted, embodiments in the specification that recite “comprising” various components are also contemplated as “consisting of” or “consisting essentially of” the recited components.

The term “channel,” “color channel,” or “optical channel”, as used herein, generally refers to a range of wavelengths. The channel may be set or determined based on particular filters which remove or filter out particular wavelengths. The terms “channel,” “color channel,” and “optical channel” can be used interchangeably.

Polymerase Chain Reaction (PCR) is a method of exponential amplification of specific nucleic acid target in a reaction mix with a nucleic acid polymerase and primers. Primers are short single stranded oligonucleotides which are complementary to the 3′ sequences of the positive and negative strand of the target sequence. The reaction mix is cycled in repeated heating and cooling steps. The heating cycle denatures or splits a double stranded nucleic acid target into single stranded templates. In the cooling cycle, the primers bind to complementary sequence on the template. After the template is primed the nucleic acid polymerase creates a copy of the original template. Repeated cycling exponentially amplifies the target 2-fold with each cycle leading to approximately a billion-fold increase of the target sequence in 30 cycles (Saiki et al 1988).

Real-Time PCR (qPCR) is a process of monitoring a PCR reaction by recording the fluorescence generated either by an intercalating dye such as SYBR Green or a target-specific reporter probe at each cycle. This is generally performed on a Real-Time PCR instrument that executes thermal cycling of the sample to complete the PCR cycles and at a specified point in each cycle measures the fluorescence of the sample in each channel through a series of excitation/emission filter sets.

Primers, or “amplification oligomers,” used herein interchangeably, refer to an oligonucleotide or nucleic acid configured to bind to another nucleic acid and facilitate one or more reactions, for example, transcription, nucleic acid synthesis, and nucleic acid amplification. A primer can be double-stranded. A primer can be single-stranded. A primer can be a forward primer or a reverse primer. A forward primer and a reverse primer can be those which bind to opposite strands of a double-stranded nucleic acid. For example, a forward primer can bind to a region of a first strand (e.g., Watson strand) derived from a nucleic acid, and a reverse primer can bind to a region of a second strand (e.g., Crick strand) derived from the nucleic acid. A forward primer may bind to a region closer to the start site of a gene relative to a reverse primer or may bind closer to the end site of a gene relative to a reverse primer. A forward primer may bind to the coding strand of a nucleic acid or may bind to the non-coding strand of a nucleic acid. A reverse primer may bind to the coding strand of a nucleic acid or may bind to the non-coding strand of a nucleic acid.

Frequently, the target-specific oligonucleotide probe is a short oligonucleotide complementary to one strand of the amplified target. The probe lacks a 3′ hydroxyl and therefore is not extendable by the DNA polymerase. TaqMan® (ThermoFisher Scientific) chemistry is a common reporter probe method used for multiplex Real-Time PCR (Holland et al. 1991). The TaqMan oligonucleotide probe is covalently modified with a fluorophore and a quenching tag (i.e., quencher). In this configuration the fluorescence generated by the fluorophore is quenched and is not detected by the real time PCR instrument. When the target of interest is present, the probe oligonucleotide base pairs with the amplified target. While bound, it is digested by the 5′ to 3′ exonuclease activity of the Taq polymerase thereby physically separating the fluorophore from the quencher and liberating signal for detection by the real time PCR instrument.

### Overview

Amplification techniques of target genes or sequences are used to determine (e.g., quantify) the initial concentration of the target genes or sequences. The initial concentration may be referred to as, or identified by, the threshold cycle (Cₜ). The Cₜ is the particular cycle number where the fluorescent signal of a qPCR amplification reaction crosses the threshold, thereby corresponding to a detectable concentration of amplicons at a particular amplification cycle (e.g., a qPCR temperature cycle). The initial period of a reaction, where none of the amplicons in the amplification have entered an exponential phase, is treated as background fluorescence. Importantly, detectable Cₜ value can subsequently be correlated to known prior concentrations to derive a concentration of the target gene or sequence, thus playing a key role in the quantification of the target gene or sequence.

Commonly, where two different amplicons are amplified (e.g., two different target genes), end point melting curve analysis is often used to determine whether a single specific amplicon is amplified, whether both amplicons were amplified, or whether neither of the amplicons was amplified. In a successful amplification, both amplicons are amplified if the targets for both are present and the Cₜ value for each of the amplicons can be calculated.

According to the various embodiments of the present disclosure, the unknown initial concentrations (e.g., Cₜ values) can be found by analyzing the amplitude curve of the multiplexed reaction, also referred to as the sum amplitude curve, which relates to the total number of amplicons per detection cycle. In some cases, validation of the sum amplitude curve may be obtained by analyzing of the melt curve (e.g., melting curve analysis).

In some cases, one of the amplification reactions is a control reaction for which the Cₜ value (e.g., concentration) is known. In other cases, concentration of both targets is unknown.

According to some embodiments of the present disclosure, methods of analyzing the sum amplitude curve (e.g., corresponding to a total detected intensity) of the multiplexed amplicons is provided, thereby enabling one to derive the Cₜ and a corresponding amplitude curve for each of the multiplexed reactions. Such methods are also compatible for the case where primers with attached fluorophores are used. As previously noted, methods according to the various embodiments of the present disclosure can be extended to multiple (e.g., more than two) amplicons.

By examining the initial amplitude of each slope, one can determine that a single amplicon is being amplified during each slope (e.g., by verifying a slope amplitude representing an exponential amplification rate). Although Cₜ points are established, one still needs to associate specific Cₜ points to a specific amplicon of the multiplexed reaction.

Instances where amplifications of the two targets occur at differing cycle numbers make it relatively easy to determine the Cₜ value for each of the reactions by using simple mathematical analysis, such as for example, by using the double derivative of the sum amplitude curve, since there is very little overlap (e.g., of the amplification slope) of the two amplification curves as further displayed by the two distinct peaks in the derivative curves.

However, in some instances, it may be difficult to determine the Cₜ value for each of the reactions by using simple mathematical analysis. Accordingly, in instances where amplifications of the two targets occur at similar cycle numbers, differentiating the amplification cycles between two or more nucleic acid targets in a PCR reaction is challenging because the curve signatures of each individual targets exhibit similar shapes. Consequently, the inability to differentiate between Cₜ values, inhibits the ability to precisely extract quantify two or more targets in multiplex PCR reactions.

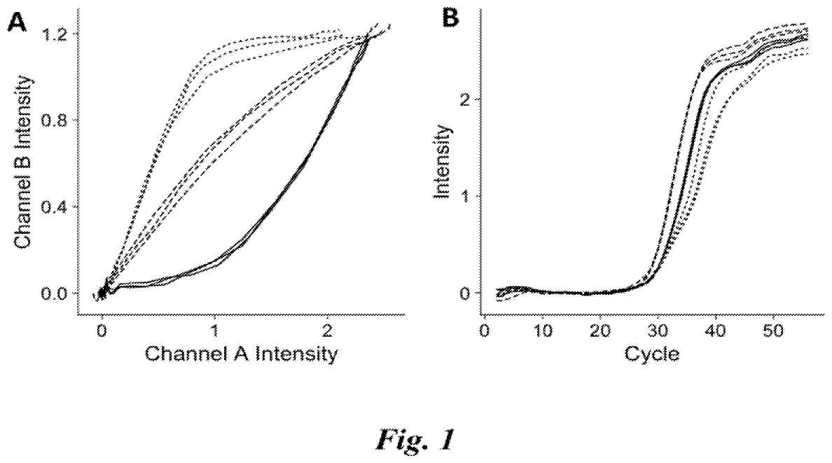

FIG. 1 show the various possible relationships between the amplitude curves for the case of a multiplexed reaction using two targets, each identified by a corresponding amplicon respectively. It is assumed that the reaction is proper, such as the correct targets are amplified at an expected efficiency (e.g. amplitude slope).

In FIG. 1B the total detected signal (e.g., total emitted fluorescence intensity detected via a single detection channel) is represented by the sum amplitude curve (sum), which corresponds to the sum of the individual amplitude curves (amplicon1) and (amplicon2), wherein amplicon1is Rhinovirus and amplicon2 is RSVA

In the case represented by FIG. 1B, the Cₜ values are similar, thus rendering determination of the Cₜ values difficult. However, a closer attention to the derivative curve shows an asymmetrical (bell) curve shape due to a skew of the two Cₜ points. Here, the double derivative curve has more peaks than the amplification curve of a single amplicon which further indicates a skew of the two Cₜ points.

Due to small variances in Cₜ values between the two targets and subsequent variance in PCR end-points between experimental conditions, differentiation (i.e., quantification) between the targets in a multiplex assay becomes challenging. The ability to accurately detect the end-point value is essential for quantification of each analyte. Thus, variances in PCR end-points makes precise quantification difficult.

However, by encoding nucleic acid targets with fluorescent probes that fluoresce in more than one fluorescent channel, a method of quantifying the presence of at least one nucleic acid analyte in a single sample volume without immobilization, separation, mass spectrometry, or melting curve analysis; thereby overcoming the inability to precisely quantify similar targets in a multiplex reactions.

FIG. 1A illustrates the parametric trajectory of the qPCR run wherein RSVA was encoded with fluorescent probes for two channels, thereby generating a different curve shape depending on the target which amplifies first. The X-axis charts the intensity in one channel, whereas the Y-axis charts the intensity in a second channel. Significantly, as the intersection of the two targets reveals end-point of the qPCR reaction.

By directly comparing the amplitude curves of one or more targets against a standard curve, the disclosed method can be used to determine absolute quantification. Similarly, the disclosed method can be used to determine relative quantification by first determining the absolute quantification of at least a first target to a standard curve before comparing a second target relative to the first amplified target. Algorithms according to the various embodiments of the present disclosure can be used to separate the different amplification curves.

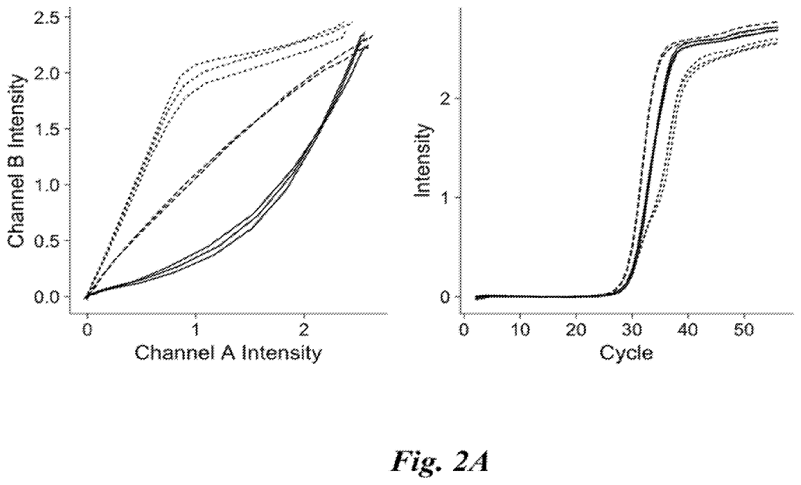

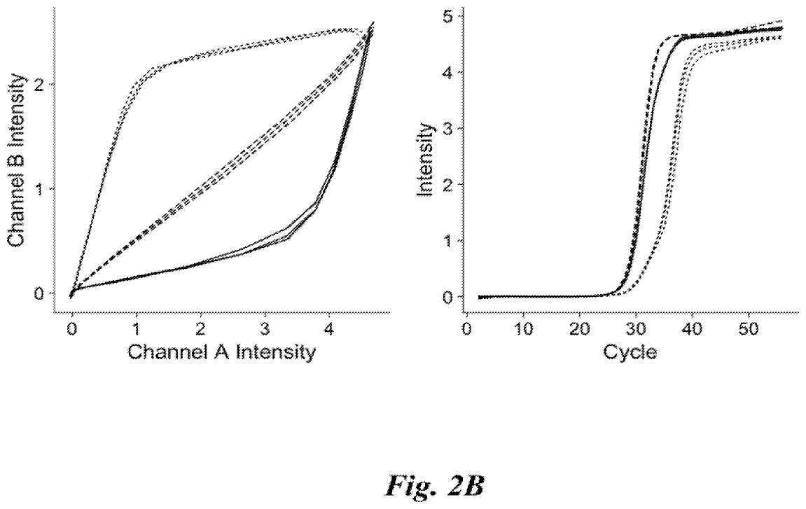

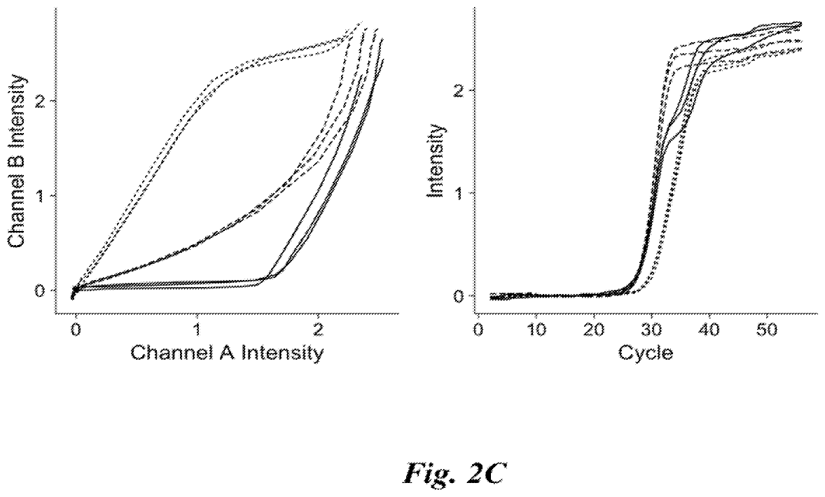

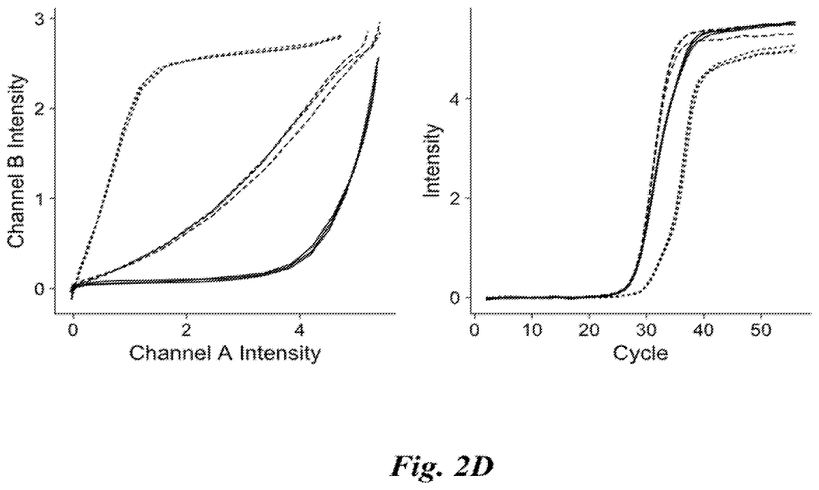

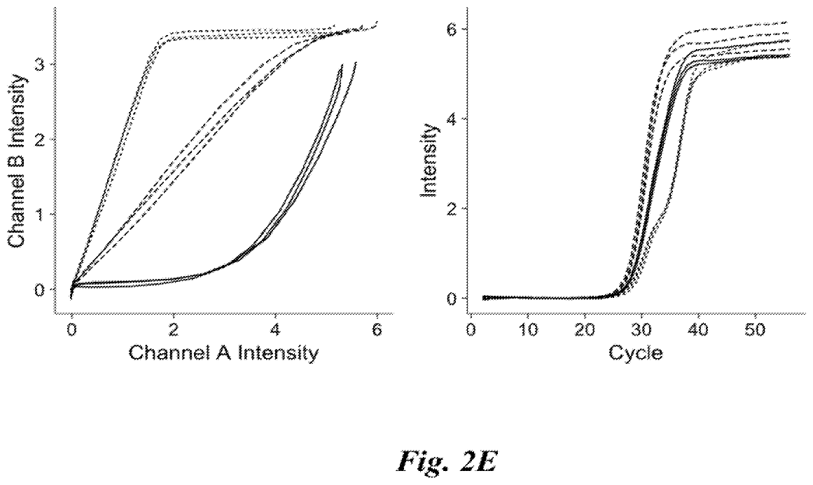

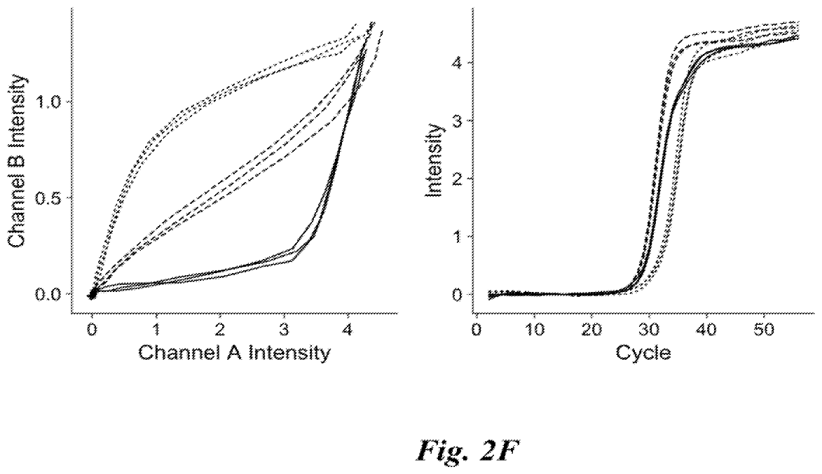

It should be noted that the although two different amplicons obtained by amplification of two different targets is presented, a multiplex reaction with two targets should not be considered as a limitation of the presented embodiments but rather an exemplary case of the inventive concept as disclosed herein. Accordingly, the method of FIG. 1A may be expanded to include many targets within an assay. FIG. 2 shows an additional 6 combinations of different combinations of targets in the same assay as FIG. 1.

Disclosed herein is a method of quantifying one or more analytes in a single sample volume without immobilization, separation, mass spectrometry, or melting curve analysis. Methods as described herein may be used to quantify 1, 2, 3, 4, 5, 6, 7, 8, 9, 10, or more analytes in a sample volume. First, a mixture may be provided comprising a plurality of nucleic acid molecules and a plurality of oligonucleotide probes. The plurality of nucleic acid molecules may be derived from, and/or may correspond with, the nucleic acid target in the sample. The plurality of oligonucleotide probes may each correspond to a different region of the nucleic acid target. The mixture may further comprise other reagents (e.g., amplification reagents) including, for example, oligonucleotide primers, dNTPs, a nucleic acid enzyme (e.g., a polymerase), and salts (e.g., Ca2+, Mg2+, etc.). Next, the mixture may be used in a quantitative Polymerase Chain Reaction (qPCR), whereby a plurality of signals may be generated. At least one of the plurality of signals may be detectable in multiple color channels. Based on the detecting, the nucleic acid target in the sample may be quantified. A signal of the plurality of the signal may be detectable in only one color channel. For example, the a first signal of the plurality of signals is detected in multiple color channels, and a second signal is detectable in only one color channel, and the analytes correlated to the first and second signals may be quantified. In another example, a first signal of the plurality of the signals is detected in a first two color channels and a second signal of the plurality of signals is detected in a second two color channels, and at least one of the channels in the first two color channels and the second two color channels is the same or substantially the same color channel. A signal may be detected or measured at least 1, 2, 3, 4, 5, 6, 7, 8, 9, 10, 11, 12, 13, 14, 15, 20, 30, 40, 50, or more channels. A signal may be detected or measured in no more than 1, 2, 3, 4, 5, 6, 7, 8, 9, 10, 11, 12, 13, 14, 15, 20, 30, 40, 50, or less channels.

The plurality of signals may be generated by one or more of the plurality of probes from the mixture. The plurality of signals may be generated by nucleic acid amplification (e.g., PCR) of the plurality of nucleic acid molecules. Nucleic acid amplification may degrade the plurality of oligonucleotide probes (e.g., by activity of a nucleic acid enzyme), thereby generating the plurality of signals. A plurality of signals may be a plurality of fluorescent signals, a plurality of chemiluminescent signals, or a combination thereof.

In multiple aspects as described herein, signals and data relating to the detection of the signals are subjected to processing in order for the signals and data to be used for subsequent steps or downstream methods. The processing may use mathematical algorithms to analyze or process the signal data. In some case, the processing may use data obtained from the instrument or detector. The processing may use data obtained from multiple channels, or a single channel. In some cases, the processing may use data from channels that are not expected to correlate with a signal from a given probe or fluorophore. For example, the data may include data obtained from a reference channel in which a background signal is obtained. The processing may use data obtained from all available channels of a given detection device.

The mathematical algorithms used for data processing may include expectation maximization, nearest neighbor analysis, basic model parameterization, Bayesian estimation, or combinations thereof. The mathematical algorithm may use a process parameter. Examples or process parameters include parameters for threshold cycles, amplitudes, or slopes.

The processing of data may comprise plotting the data. Processing the data may use plotting functions to analyze individual or multiple points such to calculate a correlation or to better visualize data. The data may be plotted as a curve. The data may be represented as a kinetic signature, wherein the signal amplitude may plotted be against a metric of time (such as cycles or seconds) or a metric that can be mathematically transformed into a metric of time (such as a frequency). The data may be fit to a variety of functions in order to derive parameters from the data. For example, a plotted data may be fit to a linear function such that a slope parameter can be derived from the data.

Processing of the data may also comprise identifying a data point as belonging to a data set. In some cases, multiple analytes are analyzed simultaneously, wherein the signal generated from analytes may comprise overlapping signals from different analytes. Processing the data may alleviate the overlapping signals or may correlate the data points to different data set in which the signal is detected via another method or alternative channel or detector.

In various aspects, reference conditions are used for comparing with data sets or for deriving reference parameters such as reference quantification parameters. Reference conditions may comprise a known concentration of a reagent or analytes. Reference conditions may comprise a known reaction condition such as the temperature or pH of a solution. For example, the reference condition may comprise a concentration, amount, or quantity of a reference nucleic acid. The reference condition with the known parameter may be used to extrapolate, interpolate or otherwise calculate a concentration, quantity, or amount of another nucleic acid in a separate sample. Reference conditions may comprise signals which may be detected or processed or as described elsewhere herein for any other signal. For example, data from the reference condition may be used to generate reference data which in turn may be parameterized by mathematical algorithms to generate reference quantification parameters. The generation of reference quantification parameters can be used to directly compare to generated quantification parameters of a data set or can be used to calculate a quantification parameter based on for example, parameterization, fitting, extrapolation, interpolation, or estimation of the data set or a parameter of the data set.

References conditions may be specific to a type of reaction. Reference conditions may comprise conditions for an amplification reaction. Examples of amplification reactions are describes elsewhere herein. Reference conditions may, for example, comprise a concentration of a polymerase or a type of polymerase. For example, reference conditions may comprise a) a primer concentration, b) a polymerase concentration, c) polymerase type, d) a reference nucleic acid concentration, e) a number of thermocycles, f) a rate of thermocycling, g) a thermocycle time length, h) a probe sequence; i) a primer sequence, or combinations thereof. Refer

In some cases, the sample further comprises an additional plurality of nucleic acid molecules and an additional plurality of oligonucleotide probes. The additional plurality of nucleic acid molecules may be derived from and/or correspond with an additional nucleic acid target. The additional plurality of oligonucleotide probes may each correspond to a different region of the additional nucleic acid target.

In various aspects, nucleic acid molecules may be quantified. The quantification may be an absolute quantification. For example, the molarity of a starting amount of a nucleic acid may be determined. This may be determined using a reference condition or amount with a known molarity of nucleic acid. The quantification may be a relative quantification. For example, a second nucleic acid may be determined to have a larger starting amount than a first nucleic acid.

A sample may be a biological sample. A sample may be derived from a biological sample. A biological sample may be, for example, blood, plasma, serum, urine, saliva, mucosal excretions, sputum, stool or tears. A biological sample may be a fluid sample. A fluid sample may be blood or plasma. A biological sample may comprise cell-free nucleic acid (e.g., cell-free RNA, cell-free DNA, etc.). A nucleic acid target may be a nucleic acid from a pathogen (e.g., virus, bacteria, etc.). A nucleic acid target may be a nucleic acid suspected of comprising one or more mutations. Assays

In some embodiments, the present disclosure provides a multiplexed assay for simultaneous amplification, detection, and or/quantification of at least one analytes in a sample. In some embodiments the methods of the disclosure may be used to detect and/or quantify at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 500, 1000, 2000, 3000, or more different target analytes in a sample. In some embodiments the methods of the disclosure may be used to detect and/or quantify at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 500, 1000, 2000, 3000, or less different target analytes a sample.

In some cases, assays may be run using the reagents in the chemical composition. Assay may use a reagent to perform a reaction. The reaction may comprise a hybridization reaction. For example, the reagent may comprise a nucleic acid and hybridize with another nucleic acid. The nucleic acid and the another nucleic acid may be complementary to one another. The reaction may comprise an extension reaction. For example, the reaction may comprise extending a nucleic molecule by the addition of a nucleotide. The reaction may comprise a polymerase chain reaction.

Methods as described herein may be performed without the use of immobilization, separation, mass spectrometry, or melting curve analysis. For example, the sample reagents and analytes may all be in solution. The analytes may be analyzed without needing to purify or physically separate the analytes from one another. Identification of the analytes may be performed without obtaining a mass of the analytes via mass spectrometry or any similar technique. Additionally, the methods may be used without observing a melting reaction and plotting the signal against a temperature. For example, an analyte may be identified without subjecting the analyte to temperature gradient in order to analyze a specific temperature in which an analyte goes through a physical or chemical change. The methods as described herein may be corroborated via techniques using immobilization, separation, mass spectrometry, or melting curve analysis.

Any number of nucleic acid targets may be detected using assays of the present disclosure. In some cases, an assay may unambiguously detect at least 3, 4, 5, 6, 7, 8, 9, 10, 11, 12, 13, 14, 15, 20, 30, 40, 50 nucleic acid targets, or more. In some cases, an assay may unambiguously detect at most 50, 40, 30, 20, 15, 14, 13, 12, 11, 10, 9, 8, 7, 6, 5, 4, or 3 nucleic acid targets. An assay may comprise any number of reactions, where the results of the reactions together identify a plurality of nucleic acid targets, in any combination of presence or absence. An assay may comprise 2, 3, 4, 5, 6, 7, 8, 9, 10 reactions, or more. Each reaction may be individually incapable of non-degenerately detecting the presence or absence of any combination of nucleic acid targets. However, the results of each reaction together may unambiguously detect the presence or absence of each of the nucleic acid targets.

Reactions may be performed in the same sample solution volume. For example, a first reaction may generate a fluorescent signal in a at least a first color channel, while a second reaction may generate a fluorescent signal in a second color channel, thereby generating two measurements for comparison. Alternatively, reactions may be performed in different sample solution volumes. For example, a first reaction may be performed in a first sample solution volume and generate a fluorescent signal in at least two channels, and a second reaction may be performed in a second sample solution volume and generate a fluorescent signal in the same color channel or a different color channel, thereby generating two measurements for comparison.

Each oligonucleotide probe may be labeled with a fluorophore. Fluorescent molecules may be excited at a wavelength at emit light at another wavelength. The fluorescent molecules may be visible to the naked human eye. The fluorescent molecules may visible or identified via spectroscopic methods such to analyze the wavelength of light that are transmitted or absorbed by a solution comprising a fluorescent molecule.

The fluorescent molecules may have a distinct or known signature of excitation or emission wavelength of electromagnetic radiation. The detection of a fluorescent molecule signature may comprise identifying an amplitude or amplitudes of signal at different wavelengths. In some cases, the fluorescent molecule signature may comprise a signal at wavelengths that overlaps with wavelengths that may be generated by reagents in the chemical composition. In some cases, the excitation wavelength of the molecule may comprise a signal that overlaps with wavelengths that may be generated by reagents in the chemical composition. In some cases, the signals of the reaction and the fluorescent molecule may be simultaneously detected. Non-limiting examples of fluorescent molecules that may be used include Alexa Fluor 350, Alexa Fluor 350, Alexa Fluor 405, Alexa Fluor 488, Alexa Fluor 532, Alexa Fluor 546, Alexa Fluor 555, Alexa Fluor 568, Alexa Fluor 594, Alexa Fluor 647, Alexa Fluor 680, Alexa Fluor 750, Cy3, Cy5, Texas Red, Fluorescein (FITC), 6-FAM, 5-FAM, HEX, JOE, TAMRA, ROX, BODIPY FL, Pacific Blue, Pacific Green, Coumarin, Oregon Green, Pacific Orange, Trimethylrhodamine (TRITC), DAPI, APC, Cyan Fluorescent Protein (CFP), Green Fluorescent Protein (GFP), Red Fluorescent Protein (RFP), Phycoerythin (PE), quantum dots (for example, Qdot 525, Qdot 565, Qdot 605, Qdot 705, Qdot 800), or derivatives thereof.

### Amplification

In some aspects, the disclosed methods comprise nucleic acid amplification. Amplification conditions may comprise thermal cycling conditions, including temperature and length in time of each thermal cycle. The use of particular amplification conditions may serve to modify the signal intensity of each signal, thereby enabling each signal to correspond to a unique combination of nucleic acid targets. Amplification may comprise using enzymes such to produce additional copies of a nucleic. The amplification reaction may comprise using oligonucleotide primers as described elsewhere herein. The oligonucleotide primers may use specific sequences to amplify a specific sequence. The oligonucleotide primers may amplify a specific sequence by hybridizing to a sequence upstream and downstream of the primers and result in amplifying the sequence inclusively between the upstream and downstream primer. The amplification reaction may comprise the use of nucleotide tri-phosphate reagents. The nucleotide tri-phosphate reagents may comprise using deoxyribo-nucleotide tri-phosphate (dNTPs). The nucleotide tri-phosphate reagents may be used as precursors to the amplified nucleic acids. The amplification reaction may comprise using oligonucleotide probes as described elsewhere herein. The amplification reaction may comprise using enzymes. Non-limiting examples of enzymes include thermostable enzymes, DNA polymerases, RNA polymerases, and reverse transcriptases. The amplification reaction may comprise generating nucleic acid molecules of a different nucleotide types. For example, a target nucleic acid may comprise DNA and an RNA molecule may be generated. In another example, an RNA molecule may be subjected to an amplification reaction and a cDNA molecule may be generated.

### Thermal Cycling

Methods of the present disclosure may comprise thermal cycling. Thermal cycling may comprise one or more thermal cycles. Thermally cycling may be performed under reaction conditions appropriate to amplify a template nucleic acid with PCR. Amplification of a template nucleic acid may require binding or annealing of oligonucleotide primer(s) to the template nucleic acid. Appropriate reaction conditions may include appropriate temperature conditions, appropriate buffer conditions, and the presence of appropriate reagents. Appropriate temperature conditions may, in some cases, be such that each thermal cycle is performed at a desired annealing temperature. A desired annealing temperature may be sufficient for annealing of an oligonucleotide probe(s) to a nucleic acid target. Appropriate buffer conditions may, in some cases, be such that the appropriate salts are present in a buffer used during thermal cycling. Appropriate salts may include magnesium salts, potassium salts, ammonium salts. Appropriate buffer conditions may be such that the appropriate salts are present in appropriate concentrations. Appropriate reagents for amplification of each member of a plurality of nucleic acid targets with PCR may include deoxyribonucleotide triphosphates (dNTPs). dNTPs may comprise natural or non-natural dNTPs including, for example, dATP, dCTP, dGTP, dTTP, dUTP, and variants thereof.

In various aspects, primer extension reactions are utilized to generate amplified product. Primer extension reactions generally comprise a cycle of incubating a reaction mixture at a denaturation temperature for a denaturation duration and incubating a reaction mixture at an elongation temperature for an elongation duration. In any of the various aspects, multiple cycles of a primer extension reaction can be conducted. Any suitable number of cycles may be conducted. For example, the number of cycles conducted may be less than about 100, 90, 80, 70, 60, 50, 40, 30, 20, 10, or 5 cycles. The number of cycles conducted may depend upon, for example, the number of cycles (e.g., cycle threshold value (CO) used to obtain a detectable amplified product (e.g., a detectable amount of amplified DNA product that is indicative of the presence of a target DNA in a nucleic acid sample). For example, the number of cycles used to obtain a detectable amplified product (e.g., a detectable amount of DNA product that is indicative of the presence of a target DNA in a nucleic acid sample) may be less than about or about 100 cycles, 75 cycles, 70 cycles, 65 cycles, 60 cycles, 55 cycles, 50 cycles, 40 cycles, 35 cycles, 30 cycles, 25 cycles, 20 cycles, 15 cycles, 10 cycles, or 5 cycles. Moreover, in some embodiments, a detectable amount of an amplifiable product (e.g., a detectable amount of DNA product that is indicative of the presence of a target DNA in a nucleic acid sample) may be obtained at a cycle threshold value (CO of less than 100, 75, 70, 65, 60, 55, 50, 45, 40, 35, 30, 25, 20, 15, 10, or 5.

The time for which an amplification reaction yields a detectable amount of amplified nucleic acid may vary depending upon the nucleic acid sample, the sequence of the target nucleic acid, the sequence of the primers, the particular nucleic acid amplification reactions conducted, and the particular number of cycles of the amplification, the temperature of the reaction, the pH of the reaction. For example, amplification of a target nucleic acid may yield a detectable amount of product indicative to the presence of the target nucleic acid at time period of 120 minutes or less; 90 minutes or less; 60 minutes or less; 50 minutes or less; 45 minutes or less; 40 minutes or less; 35 minutes or less; 30 minutes or less; 25 minutes or less; 20 minutes or less; 15 minutes or less; 10 minutes or less; or 5 minutes or less.

In some embodiments, amplification of a nucleic acid may yield a detectable amount of amplified DNA at time period of 120 minutes or less; 90 minutes or less; 60 minutes or less; 50 minutes or less; 45 minutes or less; 40 minutes or less; 35 minutes or less; 30 minutes or less; 25 minutes or less; 20 minutes or less; 15 minutes or less; 10 minutes or less; or 5 minutes or less.

### Nucleic Acid Targets

A nucleic acid target of the present disclosure may be derived from a biological sample. A biological sample may be a sample derived from a subject. A biological sample may comprise any number of macromolecules, for example, cellular macromolecules. A biological sample may be derived from another sample. A biological sample may be a tissue sample, such as a biopsy, core biopsy, needle aspirate, or fine needle aspirate. A biological sample may be a fluid sample, such as a blood sample, urine sample, or saliva sample. A biological sample may be a skin sample. A biological sample may be a cheek swab. A biological sample may be a plasma or serum sample. A biological sample may comprise one or more cells. A biological sample may be, for example, blood, plasma, serum, urine, saliva, mucosal excretions, sputum, stool or tears.

A nucleic acid target may be derived from one or more cells. A nucleic acid target may comprise deoxyribonucleic acid (DNA). DNA may be any kind of DNA, including genomic DNA. A nucleic acid target may be viral DNA. A nucleic acid target may comprise ribonucleic acid (RNA). RNA may be any kind of RNA, including messenger RNA, transfer RNA, ribosomal RNA, and microRNA. RNA may be viral RNA.

Nucleic acid targets may comprise one or more members. A member may be any region of a nucleic acid target. A member may be of any length. A member may be, for example, up to 1, 2, 3, 4, 5, 10, 20, 50, 100, 500, 1000, 5000, 10000, 50000, or 100000 nucleotides, or more. In some instances, a member may be a gene. A nucleic acid target may comprise a gene whose detection may be useful in diagnosing one or more diseases. A gene may be a viral gene or bacterial gene whose detection may be useful in identifying the presence or absence of a pathogen in a subject. In some cases, the methods of the present disclosure are useful in detecting the presence or absence or one or more infectious agents (e.g., viruses) in a subject.

Nucleic acid targets may be of various concentrations in the reaction. The nucleic acid sample may be diluted or concentrated to achieve different concentrations of nucleic acids. The concentration of the nucleic acids in the nucleic acid sample may at least 0.1 nanograms per microliter (ng/μL), 0.2 ng/μL, 0.5 ng/μL, 1 ng/μL, 2 ng/μL, 3 ng/μL, 5 ng/μL, 10 ng/μL, 20 ng/μL, 30 ng/μL, 40, ng/μL, 50 ng/μL, 100 ng/μL, 1000 ng/μL, 10000 ng/μL or more. In some cases, the concentration of the nucleic acids in the nucleic acid sample may be at most ng/μL, 0.2 ng/μL, 0.5 ng/μL, 1 ng/μL, 2 ng/μL, 3 ng/μL, 5 ng/μL, 10 ng/μL, 20 ng/μL, 30 ng/μL, 40, ng/μL, 50 ng/μL, 100 ng/μL, 1000 ng/μL, 10000 ng/μL or less.

### Sample Processing

A sample may be processed concurrently with, prior to, or subsequent to the methods of the present disclosure. A sample may be processed to purify or enrich for nucleic acids (e.g., to purify nucleic acids from a plasma sample). A sample comprising nucleic acids may be processed to purity or enrich for nucleic acid of interest.

### Nucleic Acid Enzymes

Mixtures and compositions of the present disclosure may comprise one or more nucleic acid enzymes. A nucleic acid enzyme may have exonuclease activity. A nucleic acid enzyme may have endonuclease activity. A nucleic acid enzyme may have RNase activity. A nucleic acid enzyme may be capable of degrading a nucleic acid comprising one or more ribonucleotide bases. A nucleic acid enzyme may be, for example, RNase H or RNase III. An RNase III may be, for example, Dicer. A nucleic acid may be an endonuclease I such as, for example, a T7 endonuclease I. A nucleic acid enzyme may be capable of degrading a nucleic acid comprising a non-natural nucleotide. A nucleic acid enzyme may be an endonuclease V such as, for example, an E. coli endonuclease V.

A nucleic acid enzyme may be a polymerase (e.g., a DNA polymerase). A DNA polymerase may be used. Any suitable DNA polymerase may be used, including commercially available DNA polymerases. A DNA polymerase generally refers to an enzyme that is capable of incorporating nucleotides to a strand of DNA in a template bound fashion. A polymerase may be Taq polymerase or a variant thereof. Non-limiting examples of DNA polymerases include Taq polymerase, Tth polymerase, Tli polymerase, Pfu polymerase, VENT polymerase, DEEPVENT polymerase, EX-Taq polymerase, LA-Taq polymerase, Expand polymerases, Sso polymerase, Poc polymerase, Pab polymerase, Mth polymerase, Pho polymerase, ES4 polymerase, Tru polymerase, Tac polymerase, Tne polymerase, Tma polymerase, Tih polymerase, Tfi polymerase, Platinum Taq polymerases, Hi-Fi polymerase, Tbr polymerase, Tfl polymerase, Pfutubo polymerase, Pyrobest polymerase, Pwo polymerase, KOD polymerase, Bst polymerase, Sac polymerase, Klenow fragment, and variants, modified products and derivatives thereof. For certain Hot Start Polymerase, a denaturation step at 94° C.-95° C. for 2 minutes to 10 minutes may be required, which may change the thermal profile based on different polymerases. A nucleic acid enzyme may be capable, under appropriate conditions, of degrading an oligonucleotide probe. For example, a nucleic acid enzyme may be a polymerase and comprise exo activity and degrade a probe resulting in a detectable signal. A nucleic acid enzyme may be capable, under appropriate conditions, of releasing a quencher from an oligonucleotide probe.

### Reactions

In various aspects disclosed elsewhere herein, reactions are performed. A reaction may comprise contacting nucleic acid targets with one or more oligonucleotide probes. A reaction may comprise contacting a sample solution volume (e.g., a droplet, well, tube, etc.) with a plurality of oligonucleotide probes, each corresponding to one of a plurality of nucleic acid targets, to generate a plurality of signals generated from the plurality of oligonucleotide probes. A reaction may comprise polymerase chain reaction (PCR).

### Oligonucleotide Primers

In various aspects disclosed elsewhere herein, oligonucleotide primers are used. An oligonucleotide primer (or “amplification oligomer”) of the present disclosure may be a deoxyribonucleic acid. An oligonucleotide primer may be a ribonucleic acid. An oligonucleotide primer may comprise one or more non-natural nucleotides. A non-natural nucleotide may be, for example, deoxyinosine.

An oligonucleotide primer may be a forward primer. An oligonucleotide primer may be a reverse primer. An oligonucleotide primer may be between about 5 and about 50 nucleotides in length. An oligonucleotide primer may be at least 5, 10, 11, 12, 13, 14, 15, 16, 17, 18, 19, 20, 25, 30, 35, 40, 45, or 50 base pairs in length, or more. An oligonucleotide primer may be at most 50, 45, 40, 35, 30, 25, 20, 19, 18, 17, 16, 15, 14, 13, 12, 11, 10, or 5 nucleotides in length. An oligonucleotide primer may be about 5, 10, 11, 12, 13, 14, 15, 16, 17, 18, 19, 20, 25, 30, 35, 40, 45, or 50 base pairs in length.

A set of oligonucleotide primers may comprise paired oligonucleotide primers. Paired oligonucleotide primers may comprise a forward oligonucleotide primer and a reverse oligonucleotide primer. A forward oligonucleotide primer may be configured to hybridize to a first region (e.g., a 3′ end) of a nucleic acid sequence, and a reverse oligonucleotide primer may be configured to hybridize to a second region (e.g., a 5′ end) of the nucleic acid sequence, thereby being configured to amplify the nucleic acid sequence under conditions sufficient for nucleic acid amplification. Different sets of oligonucleotide primers may be configured to amplify different nucleic acid target sequences. For example, a first set of oligonucleotide primers may be configured to amplify a first nucleic acid sequence of a given length, and a second set of oligonucleotide primers may be configured to amplify a second nucleic acid sequence of shorter length than the first nucleic acid sequence. In another example, a first set of oligonucleotide primers may be configured to amplify a first nucleic acid sequence of a given length, and a second set of oligonucleotide primers may be configured to amplify a second nucleic acid sequence of longer length than the first nucleic acid sequence.

A mixture may comprise a plurality of forward oligonucleotide primers. A plurality of forward oligonucleotide primers may be a deoxyribonucleic acid. Alternatively, a plurality of forward oligonucleotide primers may be a ribonucleic acid. A plurality of forward oligonucleotide primers may be between about 5 and about 50 nucleotides in length. A plurality of forward oligonucleotide primer may be at least 5, 10, 11, 12, 13, 14, 15, 16, 17, 18, 19, 20, 25, 30, 35, 40, 45, or 50 base pairs in length, or more. A plurality of forward oligonucleotide primer may be at most 50, 45, 40, 35, 30, 25, 20, 19, 18, 17, 16, 15, 14, 13, 12, 11, 10, or 5 nucleotides in length.

A mixture may comprise a plurality of reverse oligonucleotide primers. A plurality of reverse oligonucleotide primers may be a deoxyribonucleic acid. Alternatively, a plurality of reverse oligonucleotide primers may be a ribonucleic acid. A plurality of reverse oligonucleotide primers may be between about 5 and about 50 nucleotides in length. A plurality of reverse oligonucleotide primer may be at least 5, 10, 11, 12, 13, 14, 15, 16, 17, 18, 19, 20, 25, 30, 35, 40, 45, or 50 base pairs in length, or more. A plurality of reverse oligonucleotide primer may be at most 50, 45, 40, 35, 30, 25, 20, 19, 18, 17, 16, 15, 14, 13, 12, 11, 10, or 5 nucleotides in length.

A set of oligonucleotide primers (e.g., a forward primer and a reverse primer) may be configured to amplify a nucleic acid sequence of a given length (e.g., may hybridize to regions of a nucleic acid sequence a given distance apart). A pair of oligonucleotide primers may be configured to amplify a nucleic acid sequence of a length of at least 50, at least 75, at least 100, at least 125, at least 150, at least 175, at least 200, at least 225, at least 250, at least 275, or at least 300 base pairs (bp), or more. A pair of oligonucleotide primers may be configured to amplify a nucleic acid sequence of a length of at most 300, at most 275, at most 250, at most 225, at most 200, at most 175, at most 150, at most 125, at most 100, at most 75, or at most 50 bp, or less.

In some aspects, a mixture may include one or more synthetic (or otherwise generated to be different from the target of interest) primers for PCR reactions.

In some aspects, a mixture may be subjected to conditions sufficient to anneal an oligonucleotide primer to a nucleic acid molecule. In some aspects, a mixture may be subjected to conditions sufficient to anneal a plurality of oligonucleotide primers to a nucleic acid molecule.

In some aspects, a mixture may be subjected to conditions sufficient to anneal a plurality of oligonucleotide primers to a plurality of nucleic acid targets. The mixture may be subjected to conditions which are sufficient to denature nucleic acid molecules. Subjecting a mixture to conditions sufficient to anneal an oligonucleotide primer to a nucleic acid target may comprise thermally cycling the mixture under reaction conditions appropriate to amplify the nucleic acid target(s) with, for example, polymerase chain reaction (PCR).

Conditions may be such that an oligonucleotide primer pair (e.g., forward oligonucleotide primer and reverse oligonucleotide primer) are degraded by a nucleic acid enzyme. An oligonucleotide primer pair may be degraded by the exonuclease activity of a nucleic acid enzyme. An oligonucleotide primer pair may be degraded by the RNase activity of a nucleic acid enzyme. Degradation of the oligonucleotide primer pair may result in release of the oligonucleotide primer. Once released, the oligonucleotide primer pair may bind or anneal to a template nucleic acid.

### Oligonucleotide Probes

In various aspects disclosed elsewhere herein, oligonucleotide probes are used. Samples, mixtures, kits, and compositions of the present disclosure may comprise an oligonucleotide probe, also referenced herein as a “detection probe” or “probe”. An oligonucleotide probe may be a nucleic acid (e.g., DNA, RNA, etc.). An oligonucleotide probe may comprise a region complementary to a region of a nucleic acid target. The concentration of an oligonucleotide probe may be such that it is in excess relative to other components in a sample.

An oligonucleotide probe may comprise a non-target-hybridizing sequence. A non-target-hybridizing sequence may be a sequence which is not complementary to any region of a nucleic acid target sequence. An oligonucleotide probe comprising a non-target-hybridizing sequence may be a hairpin detection probe. An oligonucleotide probe comprising a non-target-hybridizing sequence may be a molecular beacon probe. Examples of molecular beacon probes are provided in, for example, U.S. Pat. No. 7,671,184, incorporated herein by reference in its entirety. An oligonucleotide probe comprising a non-target-hybridizing sequence may be a molecular torch. Examples of molecular torches are provided in, for example, U.S. Pat. No. 6,534,274, incorporated herein by reference in its entirety.

A sample may comprise more than one oligonucleotide probe. Multiple oligonucleotide probes may be the same or may be different. An oligonucleotide probe may be at least 5, at least 10, at least 15, at least 20, or at least 30 nucleotides in length, or more. An oligonucleotide probe may be at most 30, at most 20, at most 15, at most 10 or at most 5 nucleotides in length. In some examples, a mixture comprises a first oligonucleotide probe and one or more additional oligonucleotide probes. An oligonucleotide probe may be a nucleic acid (e.g., DNA, RNA, etc.). An oligonucleotide probe may be at least 2, 3, 4, 5, 6, 7, 8, 9, 10, 20, 30, 40, or 50 nucleotides in length, or more. An oligonucleotide probe may be at most 50, 40, 30, 20, 10, 9, 8, 7, 6, 5, 4, 3, or 2 nucleotides in length.

In some cases, 2, 3, 4, 5, 6, 7, 8, 9, 10, 11, 12, 13, 14, 15, 20, or more different oligonucleotide probes may be used. Each oligonucleotide probe may correspond to (e.g., capable of binding to) a given region of a nucleic acid target (e.g., a chromosome) in a sample. In one example, a first oligonucleotide probe is specific for a first region of a first nucleic acid target, a second oligonucleotide probe is specific for a second region of the first nucleic acid target, and a third oligonucleotide probe is specific for a third region of the first nucleic acid target. Each oligonucleotide probe may comprise a signal tag with about equal emission wavelengths. In some cases, each oligonucleotide probe comprises an identical fluorophore. In some cases, each oligonucleotide probe comprises a different fluorophore. In some case, each fluorophore is capable of being detected in a single optical channel. In other case, a fluorophore may be detected in multiple channels. In some cases, an oligonucleotide probe may have similar or the same detectable agent or fluorophore as another oligonucleotide probe in the sample. In some cases, an oligonucleotide probe may have a different detectable agent or fluorophore as compared to another oligonucleotide probe in the sample. In some cases, an oligonucleotide probe may have similar sequence or be capable or binding a similar sequence as another oligonucleotide probe in the sample. In some cases, an oligonucleotide probe may have a different sequence or be capable of binding a different sequence as compared to another oligonucleotide probe in the sample.

A probe may correspond to a region of a nucleic acid target. For example, a probe may have complementarity and/or homology to a region of a nucleic acid target. A probe may comprise a region which is complementary or homologous to a region of a nucleic acid target. A probe corresponding to a region of a nucleic acid target may be capable of binding to the region of the nucleic acid target under appropriate conditions (e.g., temperature conditions, buffer conditions. etc.). For example, a probe may be capable of binding to a region of a nucleic acid target under conditions appropriate for polymerase chain reaction. A probe may correspond to an oligonucleotide which corresponds to a nucleic acid target. For example, an oligonucleotide may be a primer with a region complementary to a nucleic acid target and a region complementary to a probe.

A probe may be provided at a specific concentration. In some cases, a second nucleic acid probe is provided at a concentration of at least about 2X, about 3X, about 4X, about 5X, about 6X, about 7X, about 8X, or more. In some cases, a second nucleic acid probe is provided at a concentration of at most about 8X, about 7X, about 6X, about 5X, about 4X, about 3X, or about 2X. In some cases, a second nucleic acid probe is provided at a concentration of about 2X, about 3X, about 4X, about 5X, about 6X, about 7X, or about 8X. X may be a concentration of a nucleic acid probe provided in the disclosed methods. In some cases, X is at least 50 nM, 100 nM, 150 nM, 200 nM, 250 nM, 300 nM, 350 nM, 400 nM, 450 nM, 500 nM, or greater. In some cases, X is at most 500 nM, 450 nM, 400 nM, 350 nM, 300 nM, 250 nM, 200 nM, 150 nM, 100 nM, or 50 nM. X may be any concentration of a nucleic acid probe.

A probe may be a nucleic acid complementary to a region of a given nucleic acid target. Each probe used in the methods and assays of the presence disclosure may comprise at least one fluorophore. A fluorophore may be selected from any number of fluorophores. A fluorophore may be selected from three, four, five, six, seven, eight, nine, or ten fluorophores, or more. One or more oligonucleotide probes used in a single reaction may comprise the same fluorophore. In some cases, all oligonucleotide probes used in a single reaction comprise the same fluorophore. Each probe may, when excited and contacted with its corresponding nucleic acid target, generate a signal. A signal may be a fluorescent signal. A plurality of signals may be generated from one or more probes.

An oligonucleotide probe may have less than 50%, 40%, 30%, 20%, 10%, 5%, or 1% complementarity to any member of a plurality of nucleic acid targets. An oligonucleotide probe may have no complementarity to any member of the plurality of nucleic acid targets.

An oligonucleotide probe may comprise a detectable label. A detectable label may be a chemiluminescent label. A detectable label may comprise a fluorescent label. A detectable label may comprise a fluorophore. A fluorophore may be, for example, FAM, TET, HEX, JOE, Cy3, or Cy5. A fluorophore may be FAM. A fluorophore may be HEX. An oligonucleotide probe may further comprise one or more quenchers. A quencher may inhibit signal generation from a fluorophore. A quencher may be, for example, TAMRA, BHQ-1, BHQ-2, or Dabcy. A quencher may be BHQ-1. A quencher may be BHQ-2.

### Signal Generation

Thermal cycling may be performed such that one or more oligonucleotide probes are degraded by a nucleic acid enzyme. An oligonucleotide probe may be degraded by the exonuclease activity of a nucleic acid enzyme. An oligonucleotide probe may generate a signal upon degradation. In some cases, an oligonucleotide probe may generate a signal only if at least one member of a plurality of nucleic acid targets is present in a mixture.

A reaction may generate one or more signals. A reaction may generate a cumulative intensity signal comprising a sum of multiple signals. A signal may be a chemiluminescent signal. A signal may be a fluorescent signal. A signal may be generated by an oligonucleotide probe. For example, excitation of a hybridization probe comprising a luminescent signal tag may generate a signal. A signal may be generated by a fluorophore. A fluorophore may generate a signal upon release from a hybridization probe. A reaction may comprise excitation of a fluorophore. A reaction may comprise signal detection. A reaction may comprise detecting emission from a fluorophore.

A signal may be a fluorescent signal. A signal may correspond to a fluorescence intensity level. Each signal measured in the methods of the present disclosure may have a distinct fluorescence intensity value, thereby corresponding to the presence of a unique combination of nucleic acid targets. A signal may be generated by one or more oligonucleotide probes. The number of signals generated in an assay may correspond to the number of oligonucleotide probes and nucleic acid targets present.

N may be a number of signals detected in a single optical channel in an assay of the present disclosure. N may be at least 2, 3, 4, 5, 6, 7, 8, 9, 10, 11, 12, 13, 14, 15, 20, 25, 30, 40, 50 or more. N may be at most 50, 40, 30, 24, 20, 15, 14, 13, 12, 11, 10, 9, 8, 7, 6, 5, 4, 3, or 2. N may be 2, 3, 4, 5, 6, 7, 8, 9, 10, 11, 12, 13, 14, 15, 20, 25, 30, 40, or 50.

As will be recognized and is described elsewhere herein, sets of signals may be generated in multiple different optical channels, where each set of signals is detected in a single optical channel, thereby significantly increasing the number of nucleic acid targets that can be measured in a single reaction. In some cases, two sets of signals are detected in a single reaction. Each set of signals detected in a reaction may comprise the same number of signals, or different numbers of signals.

In some cases, a signal may be generated simultaneous with hybridization of an oligonucleotide probe to a region of a nucleic acid. For example, an oligonucleotide probe (e.g., a molecular beacon probe or molecular torch) may generate a signal (e.g., a fluorescent signal) following hybridization to a nucleic acid. In some cases, a signal may be generated subsequent to hybridization of an oligonucleotide probe to a region of a nucleic acid, following degradation of the oligonucleotide probe by a nucleic acid enzyme.

In cases where an oligonucleotide probe comprises a signal tag, the oligonucleotide probe may be degraded when bound to a region of an oligonucleotide primer, thereby generating a signal. For example, an oligonucleotide probe (e.g., a TaqMan® probe) may generate a signal following hybridization of the oligonucleotide probe to a nucleic acid and subsequent degradation by a polymerase (e.g., during amplification, such as PCR amplification). An oligonucleotide probe may be degraded by the exonuclease activity of a nucleic acid enzyme.

An oligonucleotide probe may comprise a quencher and a fluorophore, such that the quencher is released upon degradation of an oligonucleotide probe, thereby generating a fluorescent signal. Thermal cycling may be used to generate one or more signals. Thermal cycling may generate at least 1, 2, 3, 4, 5, 6, 7, 8, 9, 10 signals, or more. Thermal cycling may generate at most 10, 9, 8, 7, 6, 5, 4, 3, 2, or 1 signal. Multiple signals may be of the same type or of different types. Signals of different types may be fluorescent signals with different fluorescent wavelengths. Signals of different types may be generated by detectable labels comprising different fluorophores. Signals of the same type may be of different intensities (e.g., different intensities of the same fluorescent wavelength). Signals of the same type may be signals detectable in the same color channel. Signals of the same type may be generated by detectable labels comprising the same fluorophore. Detectable labels comprising the same fluorophore may generate different signals by nature of being at different concentrations, thereby generating different intensities of the same signal type.

Although fluorescent probes have been used to illustrate this principle, the disclosed methods are equally applicable to any other method providing a quantifiable signal, including an electrochemical signal, chemiluminescent signals, magnetic particles, and electrets structures exhibiting a permanent dipole.

In certain portions of this disclosure, the signal may be a fluorescent signal. For example, like fluorescent signals, any of the electromagnetic signals described above may also be characterized in terms of a wavelength, whereby the wavelength of a fluorescent signal may also be described in terms of color. The color may be determined based on measuring intensity at a particular wavelength or range of wavelengths, for example by determining a distribution of fluorescent intensity at different wavelengths and/or by utilizing a band pass filter to determine the fluorescence intensity within a particular range of wavelengths.

The presence or absence of one or more signals may be detected. One signal may be detected, or multiple signals may be detected. Multiple signals may be detected simultaneously. Alternatively, multiple signals may be detected sequentially. A signal may be detected throughout the process of thermal cycling, for example, at the end of each thermal cycle. The signals may be detected in a multi-channel detector. For example, the signal may be observed using a detector that can observe a signal in multiple ranges of wavelengths simultaneously, substantially simultaneously, or sequentially. The signal may be observable in at least 1, 2, 3, 4, 5, 6, 7, 8, 9, 10, or more channels. The signal may be observable in no more than 10, 9, 8, 7, 6, 5, 4, 3, 2, or less channels.

In some cases, the signal intensity increases with each thermal cycle. The signal intensity may increase in a sigmoidal fashion. The presence of a signal may be correlated to the presence of at least one member of a plurality of target nucleic acids. Correlating the presence of a signal to the presence of at least one member of a plurality of target nucleic acids may comprise establishing a signal intensity threshold. A signal intensity threshold may be different for different signals. Correlating the presence of a signal to the presence of at least one member of a plurality of target nucleic acids may comprise determining whether the intensity of a signal increases beyond a signal intensity threshold. In some examples, the presence of a signal may be correlated with the presence of at least one of all members of a plurality of target nucleic acids. In other examples, the presence of a first signal may be correlated with the presence of at least one of a first subset of members of a plurality of target nucleic acids, and the presence of a second signal may be correlated with the presence of at least one of a second subset of members of a plurality of target nucleic acids.

The presence of a signal may be correlated to the presence of a nucleic acid target. The presence of least 1, 2, 3, 4, 5, 6, 7, 8, 9, 10, or more signals may be correlated with the presence of at least one of at least 1, 2, 3, 4, 5, 6, 7, 8, 9, 10, or more nucleic acid targets. The absence of a signal may be correlated with the absence of corresponding nucleic acid targets. The absence of least 1, 2, 3, 4, 5, 6, 7, 8, 9, 10, or more signals may be correlated with the absence of each of at least 1, 2, 3, 4, 5, 6, 7, 8, 9, 10, or more nucleic acid target molecules.

### Kits

The present disclosure also provides kits for analysis. Kits may comprise one or more oligonucleotide probes. Oligonucleotide probes may be lyophilized. Different oligonucleotide probes may be present at different concentrations in a kit. Oligonucleotide probes may comprise a fluorophore and/or one or more quenchers.

Kits may comprise one or more sets of oligonucleotide primers (or “amplification oligomers”) as described herein. A set of oligonucleotide primers may comprise paired oligonucleotide primers. Paired oligonucleotide primers may comprise a forward oligonucleotide primer and a reverse oligonucleotide primer. A set of oligonucleotide primers may be configured to amplify a nucleic acid sequence corresponding to particular targets. For example, a forward oligonucleotide primer may be configured to hybridize to a first region (e.g., a 3′ end) of a nucleic acid sequence, and a reverse oligonucleotide primer may be configured to hybridize to a second region (e.g., a 5′ end) of the nucleic acid sequence, thereby being configured to amplify the nucleic acid sequence. Different sets of oligonucleotide primers may be configured to amplify nucleic acid sequences. In one example, a first set of oligonucleotide primers may be configured to amplify a first nucleic acid sequence, and a second set of oligonucleotide primers may be configured to amplify a second nucleic acid sequence. Oligonucleotide primers configured to amplify nucleic acid molecules may be used in performing the disclosed methods. In some cases, all of the oligonucleotide primers in a kit are lyophilized.

Kits may comprise one or more nucleic acid enzymes. A nucleic acid enzyme may be a nucleic acid polymerase. A nucleic acid polymerase may be a deoxyribonucleic acid polymerase (DNase). A DNase may be a Taq polymerase or variant thereof. A nucleic acid enzyme may be a ribonucleic acid polymerase (RNase). An RNase may be an RNase III. An RNase III may be Dicer. The nucleic acid enzyme may be an endonuclease. An endonuclease may be an endonuclease I. An endonuclease I may be a T7 endonuclease I. A nucleic acid enzyme may be capable of degrading a nucleic acid comprising a non-natural nucleotide. A nucleic acid enzyme may be an endonuclease V such as, for example, an E. coli endonuclease V. A nucleic acid enzyme may be a polymerase (e.g., a DNA polymerase). A polymerase may be Taq polymerase or a variant thereof. A nucleic acid enzyme may be capable, under appropriate conditions, of degrading an oligonucleotide probe. A nucleic acid enzyme may be capable, under appropriate conditions, of releasing a quencher from an oligonucleotide probe. Kits may comprise instructions for using any of the foregoing in the methods described herein.

Kits provided herein may be useful in, for example, calculating at least first and second sums, each being a sum of multiple target signals corresponding with a first and second target nucleic acid.

### Systems

Methods as disclosed herein may be performed using a variety of systems. The systems may be configured such the steps of the method may be performed. For example, the systems may comprise a detector for the detection of signals as described elsewhere herein. The system may comprise a processor configured to process, receive, plot, or otherwise represent the data obtained from the detector. The processor may be configured to process the data as described elsewhere herein.

The present disclosure provides computer systems that are programmed to implement methods of the disclosure. The computer system can perform various aspects of the present disclosure. The computer system can be an electronic device of a user or a computer system that is remotely located with respect to the electronic device. The electronic device can be a mobile electronic device.

The computer system may include a central processing unit (CPU, also “processor” and “computer processor” herein), which can be a single core or multi core processor, or a plurality of processors for parallel processing. The computer system may include memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices are in communication with the CPU through a communication bus (solid lines), such as a motherboard. The storage unit can be a data storage unit (or data repository) for storing data. The computer system can be operatively coupled to a computer network (“network”) with the aid of the communication interface. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing. The network, in some cases with the aid of the computer system, can implement a peer-to-peer network, which may enable devices coupled to the computer system to behave as a client or a server.

The CPU can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the system can be included in the circuit. In some cases, the circuit is an application specific integrated circuit (ASIC).

The storage unit can store files, such as drivers, libraries and saved programs. The storage unit can store user data, e.g., user preferences and user programs. The computer system in some cases can include one or more additional data storage units that are external to the computer system, such as located on a remote server that is in communication with the computer system through an intranet or the Internet.

The computer system can communicate with one or more remote computer systems through the network. For instance, the computer system can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system via the network.

Methods as described herein can be implemented by way of machine (e.g., computer processor) executable code stored on an electronic storage location of the computer system, such as, for example, on the memory or electronic storage unit. The machine executable or machine readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system can include or be in communication with an electronic display that comprises a user interface (UI) for providing, for example, plots of data, plots of kinetic signatures, information relating to signal amplitude, Examples of UIs include, without limitation, a graphical user interface (GUI) and web-based user interface.

Methods and systems of the present disclosure can be implemented by way of one or more algorithms. An algorithm can be implemented by way of software upon execution by the central processing unit. The algorithm can, for example, parameterize data points or fit data point to specified mathematical functions, in order to quantify analytes.

### EXAMPLES

### Example 1

### Quantitation Through Parametric Trajectories

When examining the kinetic signature of an analyte in a PCR reaction, one can determine that a single amplicon is being amplified during each slope (e.g., by verifying a slope amplitude representing an exponential amplification rate). Thus, in instances where the amplifications of two targets (i.e., multiplex) occurs at differing cycle numbers, it relatively easy to determine the Cₜ value for each of the reactions by using simple mathematical analysis, such as for example, by using the double derivative of the sum amplitude curve—since there is very little overlap (e.g., of the amplification slope). However, in instances where the Cₜ values are similar, differentiation (i.e., quantification) between the targets in a multiplex assay becomes challenging.

Disclosed herein is a method of quantifying the presence of at least one nucleic acid analyte in a single sample volume using a fluorescent probe that fluoresces in more than one fluorescent channels, thereby generating two unique curve signatures which may be used to differentiate amplification cycles and quantify one or more targets in a PCR reactions.

According to some embodiments of the present disclosure, methods of analyzing the sum amplitude curve (e.g., corresponding to a total detected intensity) of multiplexed amplicons enables one to derive the Cₜ and a corresponding amplitude curve for each of the multiplexed reactions. Such methods may be used to accurately quantify of the presence of at least one nucleic acid analyte in a single sample volume without immobilization, separation, mass spectrometry, or melting curve analysis.

The method may use at least one fluorophore is measurable in at least two distinct color channels. As previously noted, methods according to the various embodiments of the present disclosure can be extended to multiple (e.g., more than two) amplicons.

FIG. 1 show the various possible relationships between the amplitude curves (i.e., kinetic signatures) for the case of a multiplexed reaction using two targets, each identified by a corresponding amplicon respectively. It is assumed that the reaction is proper, such as the correct targets are amplified at an expected efficiency (e.g. amplitude slope).

In FIG. 1B the total detected signal (e.g., total emitted fluorescence intensity detected via a single detection channel) is represented by the sum amplitude curve (sum), which corresponds to the sum of the individual amplitude curves (amplicon1) and (amplicon2), wherein amplicon1 is Rhinovirus and amplicon2 is RSVA.

FIG. 1B plots the intensity values of two targets (Rhinovirus and RSVA) in a single-channel multiplex reaction, wherein each of the targets contained various concentrations of template. The long dash denotes experimental conditions where 5,000 copies of each template were present. The short dash denotes experimental conditions where 5,000 copies of RSVA and 50 copies of Rhinovirus were present. The solid line denotes experimental conditions where 50 copies of RSVA and 5,000 copies of Rhinovirus were present. Each the three replicates signifies data extracted from three separate wells (i.e., data is aggregated from three separate amplification events). Significantly, the long dash data illustrates amplification events where both Rhinovirus and RSVA targets began to amplify at the same time. The short dash data illustrates amplification events where RSVA began to amplify before Rhinovirus, and the sold line data illustrates amplification events where Rhinovirus began to amplify before RSVA.

In the case represented by FIG. 1B, the Cₜ values are similar and end-points are similar, thus rendering determination of the Cₜ values difficult. Accordingly, where amplifications of the two targets occur at similar cycle numbers, differentiating the amplification cycles between two or more nucleic acid targets in a PCR reaction is challenging because the curve signatures of each individual targets exhibit similar shapes. Consequently, the resulting kinetic signatures relating to two or more targets in a multiplex PCR will inhibit the ability to precisely extract quantify two or more targets in multiplex PCR reactions. Closer analysis to the derivative curve shows an asymmetrical (bell) curve shape due to a skew of the two Cₜ points. Here, the double derivative curve has more peaks than the amplification curve of a single amplicon which further indicates a skew of the two Cₜ points.

The small variances in Cₜ values has an impact to PCR end-points. Significantly, accurate determination of end-points values is essential for determination of the identity and starting quantity of each analyte, as end-point values can be decoded to indicate the presence or absence of analytes. Thus, variances in PCR end-points inhibits precise quantification.

However, by encoding nucleic acid targets with fluorescent probes that fluoresce in more than one fluorescent channel, a method of quantifying the presence of at least one nucleic acid analyte in a single sample volume without immobilization, separation, mass spectrometry, or melting curve analysis; thereby overcoming the inability to precisely quantify similar targets in a multiplex reaction as illustrated in FIG. 1B

FIG. 1A exemplifies a situation where RSVA was encoded with fluorescent probes for two channels, thereby generating a different curve shape depending on the target which amplifies first. In an exemplary embodiment, Rhinovirus and RSVA were amplified in a PCR reaction with varying input concentrations of template. Again, the long dash denotes experimental conditions where 5,000 copies of each template were present. The short dash denotes experimental conditions where 5,000 copies of RSVA and 50 copies of Rhinovirus were present. The solid line denotes experimental conditions where 50 copies of RSVA and 5,000 copies of Rhinovirus were present.

FIG. 1A illustrates the parametric trajectory of the qPCR run, whereby the X-axis charts the intensity in one channel and the Y-axis charts the intensity in a second channel. The long dash data illustrates amplification events where both Rhinovirus and RSVA targets began to amplify at the same time. The short dash data illustrates amplification events where RSVA began to amplify before Rhinovirus, and the sold line data illustrates amplification events where Rhinovirus began to amplify before RSVA. Significantly, as the intersection of the two targets reveals end-point of the qPCR reaction.

Algorithms according to the various embodiments of the present disclosure can be used to separate the different amplification curves. By directly comparing the amplitude curves of one or more targets against a standard curve, method can be used to determine absolute quantification. Similarly, the method can be used to determine relative quantification by first determining the absolute quantification of at least a first target to a standard curve before comparing a second target relative to the first amplified target. Various methods may include expectation maximization, nearest neighbor analysis, basic model parameterization, Bayesian estimation, or others.

It should be noted that the although two different amplicons obtained by amplification of two different targets is presented, a multiplex reaction with two targets should not be considered as a limitation of the presented embodiments but rather an exemplary case of the inventive concept as disclosed herein. Accordingly, the method of FIG. 1A may be expanded to include many targets within an assay. FIG. 2 shows an additional 6 combinations of different combinations of targets in the same assay as FIG. 1.

### Example 2

### Twelve Targets

In an exemplary method, a 12-plex assay is constructed. Of the twelve nucleic acid targets, eight of the twelve nucleic acid targets are specifically encoded with fluorescent probes that fluoresce in more than one fluorescent channel. The targets are amplified concurrently, thereby generating at least two unique sets of curve signatures results for every combination of targets amplified.

FIG. 2A illustrates experimental data where Metapneumovirus and FluB are present. FIG. 2B illustrates experimental data where Metapneumovirus and Adenovirus are present. FIG. 2C illustrates experimental data where PIV1 and PIV3 are present. FIG. 2D illustrates experimental data where PIV 1 and PIV2 are present. FIG. 2E illustrates experimental data where PIV3 and PIV3 are present. FIG. 2F illustrates experimental data where RSVA and RSVB are present.

### Example 3

### Quantitation Through Channel Cross-Correlation

Another method for quantifying multiple targets could involve using cross correlation between channels to determine the cycle at which a target could amplify. Various correlational methods could be used, in conjunction with or without other models such as a decomposition of underlying sigmoidal curves.

This method need not be limited to the exemplary PCR examples and could be used in other types of assays leveraging multiple time-based signals, as an example ELISA with multiple colors and/or spot locations. This need not be limited to time based signals, for example could use spatial separation as a dimension.

1. A method of quantifying at least a first and a second nucleic acid in a sample, the method comprising:
a. providing a mixture comprising:
i. said first nucleic acid and said second nucleic acid;
ii. a first detection probe configured to generate a first signal when in the presence of said first nucleic acid and when subjected to reaction conditions;
iii. a second detection probe configured to generate a second signal when in the presence of said second nucleic acid and when subjected to reaction conditions;
b. subjecting said mixture to said reaction conditions thereby generating said first and second signal;
c. measuring (i) an intensity of said first signal in first range of wavelengths, (ii) an intensity of said first signal in said second range of wavelengths, (iii) an intensity of said second signal in a third range of wavelengths;
d. generating a first data set derived from said intensity (i) and said intensity (ii) measured in c) and generating a second data set derived from said intensity (iii) measured in c); and
e. processing said generated first data set and said generated second data set, wherein said processing uses reference quantification parameters derived from a reference data set(s) to generate quantification parameters of said generated first and second data set, wherein said reference data set(s) corresponds to a reference condition(s), wherein said reference condition(s) comprises a quantity of reference nucleic acid(s), thereby quantifying said first and second nucleic acid.

2. The method of claim 1, wherein the method does not comprise immobilization, separation, mass spectrometry, or melting curve analysis.

3. The method of claim 1, wherein c) further comprises measuring an intensity of said first or second signal is (iii) a third range of wavelengths.

4. The method of claim 1, wherein subjecting said mixture to said reaction conditions comprises applying electromagnetic radiation to said mixture.

5. The method of claim 1, wherein said measuring comprises detecting said first or second signal using a multi-channel detector.

6. The method of claim 1, wherein said first or second signal comprises electromagnetic radiation.

7. The method of claim 6, wherein said electromagnetic radiation comprises a wavelength of electromagnetic radiation.

8. The method of claim 6, wherein said electromagnetic radiation comprises a plurality of wavelengths of electromagnetic radiation.

9. The method of claim 1, wherein said first or second signal is generated by fluorescence emission.

10. The method of claim 1, wherein said first or second signal is generated by chemiluminescence.

11. The method of claim 1, wherein said first range of wavelengths and said second range of wavelengths comprise a same wavelength.

12. The method of claim 1, wherein said first range of wavelengths and said third range of wavelengths comprise a same wavelength.

13. The method of claim 1, wherein said first range of wavelengths and said second range of wavelengths do not comprise a same wavelength.

14. The method of claim 1, wherein said first range of wavelengths and said third range of wavelengths do not comprise a same wavelength.

15. The method of claim 1, wherein said detection probe is configured to anneal to said at least one nucleic acid.

16. The method of claim 1, wherein said detection probe is configured to generate said signal upon degradation of a portion of said detection probe.

17. The method of claim 1, wherein said detection probe comprises a fluorophore or dye.

18. The method of claim 1, wherein said mixture further comprises an amplification oligomer.

19. The method of claim 18, wherein said amplification oligomer comprises a sequence complementary to a portion of the sequence of said at least said nucleic acid.

20. The method of claim 1, wherein said reaction conditions comprise conditions for a DNA extension reaction.

21. The method of claim 20, wherein said DNA extension reaction is a polymerase chain reaction (PCR).

22. The method of claim 21, wherein said polymerase chain reaction is a quantitative polymerase chain reaction (qPCR).

23. The method of claim 1, wherein said processing comprises using a mathematical algorithm.

24. The method of claim 23, wherein said mathematical algorithm comprises expectation maximization, nearest neighbor analysis, basic model parameterization or Bayesian estimation.

25. The method of claim 23, wherein said mathematical algorithm comprises a process parameter.

26. The method of claim 25, wherein said process parameter comprises a) a threshold cycle, b) an amplitude, or c) a slope.

27. The method of claim 1, wherein said processing comprises fitting said first or second data set to a curve.

28. The method of claim 1, wherein said first or second data set is plotted as a curve.

29. The method of claim 1, wherein said first or second data set is a kinetic signature.

30. The method of claim 1, wherein said reference condition(s) comprises an amplification reaction condition.

31. The method of claim 1, wherein said reference conditions comprise a) a temperature, b) a pH, c) a concentration of said reference nucleic acid, or a combination thereof.

32. The method of claim 1, wherein said reference data set corresponds with a data set generated by amplification of said reference nucleic acid under amplification parameters, wherein said reference data set is indicative of an amplification parameter comprising: a) a primer concentration, b) a polymerase concentration, c) polymerase type, d) a reference nucleic acid concentration, e) a number of thermocycles, f) a rate of thermocycling, g) a thermocycle time length, h) a probe sequence; i) a primer sequence, or combinations thereof.

33. The method of claim 1, wherein said quantifying comprises calculating an absolute quantification.

34. The method of claim 1, wherein said reference data set is generated using predetermined concentrations of said reference nucleic acid.

35. The method of claim 1, wherein said at least one nucleic acid is derived from a biological sample.

36. The method of claim 35, wherein the biological sample is blood or plasma.

37. The method of claim 35, wherein the biological sample is derived from a virus.

38. The method of claim 1, wherein said first or second nucleic acid comprises DNA.

39. The method of claim 38, wherein said DNA comprises genomic DNA.

40. The method of claim 1, wherein said first or second nucleic acid comprises RNA.

41. The method of claim 40, wherein said RNA comprises mRNA.

42. The method of claim 1, further comprising in c), measuring an intensity of said second signal in (iv) a fourth range of wavelengths, and further comprising in d) generating said second data derived from said intensity iii) and said intensity of said second signal in said fourth range of wavelengths.

43. The method of claim 42, wherein said fourth range of wavelength is the same as said first range of wavelength or said second range of wavelengths

44. The method of claim 1, wherein said third range of wavelengths is the same as said first range of wavelength or said second range of wavelengths.

45. The method of claim 1, wherein said processing comprises identifying data points as corresponding to said first data set.

46. The method of claim 1, wherein said processing comprises identifying data points as corresponding to said second data set.

47. The method of claim 1, wherein said quantifying comprises calculating a relative quantification.

48. The method of claim 47, wherein said relative quantification is generated by comparing said first data set and said second data set.

49. A method of quantifying at least one nucleic acid in a sample volume, the method comprising:
a. providing a mixture comprising:
i. said at least one nucleic acid;
ii. a first detection probe configured to generate a signal when in the presence of said at least one nucleic acid and when subjected to reaction conditions;
b. subjecting said mixture to said reaction conditions, thereby generating said signal;
c. measuring (i) an intensity of said signal in a first range of wavelengths and (ii) an intensity of said signal in a second range of wavelengths;
d. generating a data set derived from said intensities measured in c); and
e. processing said generated data set, wherein said processing uses reference quantification parameters derived from a reference data set(s) to calculate quantification parameters of said generated data set, wherein said reference data set(s) corresponds to a reference condition(s), wherein said reference condition(s) comprises a quantity of reference nucleic acid(s), thereby quantifying said at least one nucleic acid.

50. The method of claim 49, wherein the method does not comprise immobilization, separation, mass spectrometry, or melting curve analysis.

51. The method of claim 49, wherein c) further comprises measuring (iii) an intensity of said signal in a third range of wavelengths.

52. The method of claim 49, wherein subjecting said mixture to said reaction conditions comprises applying electromagnetic radiation to said mixture.

53. The method of claim 49, wherein said measuring comprises detecting said signal using a multi-channel detector.

54. The method of claim 49, wherein said signal comprises electromagnetic radiation.

55. The method of claim 54, wherein said electromagnetic radiation comprises a wavelength of electromagnetic radiation.

56. The method of claim 54, wherein said electromagnetic radiation comprises a plurality of wavelengths of electromagnetic radiation.

57. The method of claim 49, wherein said signal is generated by fluorescence emission.

58. The method of claim 49, wherein said signal is generated by chemiluminescence.

59. The method of claim 49, wherein said first range of wavelengths and said second range of wavelengths comprise a same wavelength.

60. The method of claim 49, wherein said first range of wavelengths and said second range of wavelengths do not comprise a same wavelength.

61. The method of claim 49, wherein said detection probe is configured to anneal to said at least one nucleic acid.

62. The method of claim 49, wherein said detection probe is configured to generate said signal upon degradation of a portion of said detection probe.

63. The method of claim 49, wherein said detection probe comprises a fluorophore or dye.

64. The method of claim 49, wherein said mixture further comprises an amplification oligomer.

65. The method of claim 64, wherein said amplification oligomer comprises a sequence complementary to a portion of the sequence of said at least said nucleic acid.

66. The method of claim 49, wherein said reaction conditions comprise conditions for a DNA extension reaction.

67. The method of claim 66, wherein said DNA extension reaction is a polymerase chain reaction (PCR).

68. The method of claim 67, wherein said polymerase chain reaction is a quantitative polymerase chain reaction (qPCR).

69. The method of claim 49, wherein said processing comprises using a mathematical algorithm.

70. The method of claim 69, wherein said mathematical algorithm comprises expectation maximization, nearest neighbor analysis, basic model parameterization or Bayesian estimation.

71. The method of claim 69, wherein said mathematical algorithm comprises a process parameter.

72. The method of claim 71, wherein said process parameter comprises a) a threshold cycle, b) an amplitude, or c) a slope.

73. The method of claim 49, wherein said processing comprises fitting said generated data set to a curve.

74. The method of claim 49, wherein said data set is plotted as a curve.

75. The method of claim 49, wherein said data set comprises a kinetic signature.

76. The method of claim 49, wherein said reference condition(s) comprises an amplification reaction condition.

77. The method of claim 49, wherein said reference conditions comprise a) a temperature, b) a pH, c) a concentration of said reference nucleic acid, or a combination thereof.

78. The method of claim 49, wherein said reference data set corresponds with a data set generated by amplification of said reference nucleic acid under amplification parameters, wherein said reference data set is indicative of an amplification parameter comprising: a) a primer concentration, b) a polymerase concentration, c) polymerase type, d) a reference nucleic acid concentration, e) a number of thermocycles, f) a rate of thermocycling, g) a thermocycle time length, h) a probe sequence; i) a primer sequence, or combinations thereof.

79. The method of claim 49, wherein said quantifying comprises calculating an absolute quantification.

80. The method of claim 49, wherein said reference data set is generated using predetermined concentrations of said reference nucleic acid.

81. The method of claim 49, wherein said at least one nucleic acid is derived from a biological sample.

82. The method of claim 81, wherein said biological sample is blood or plasma.

83. The method of claim 81, wherein said biological sample is derived from a virus.

84. The method of claim 49, wherein said at least one nucleic acid comprises DNA.

85. The method of claim 84, wherein said DNA comprises genomic DNA.

86. The method of claim 49, wherein said at least one nucleic acid comprises RNA.

87. The method of claim 86, wherein said RNA comprises mRNA.

88. A system, comprising a controller comprising or capable of accessing, computer readable media comprising non-transitory computer-executable instructions which, when executed by at least one electronic processor perform a method comprising:
a. providing a mixture comprising:
i. said at least one nucleic acid;
ii. at least a first detection probe configured to generate a signal when in the presence of said at least one nucleic acid and when subjected to reaction conditions;
b. subjecting said mixture to said reaction conditions, thereby generating said signal;
c. measuring (i) an intensity of said signal in a first range of wavelengths and (ii) an intensity of said signal in a second range of wavelengths;
d. generating a data set derived from said intensities measured in c); and
e. processing said generated data set, wherein said processing uses reference quantification parameters derived from a reference data set(s) to calculate quantification parameters of said generated data set, wherein said reference data set(s) corresponds to a reference condition(s), wherein said reference condition(s) comprises a quantity of reference nucleic acid, thereby quantifying said at least one nucleic acid.

89. The system of claim 88, wherein the method does not comprise immobilization, separation, mass spectrometry, or melting curve analysis.

90. The system of claim 88, wherein c) further comprises measuring an intensity of said signal is (iii) a third range of wavelengths.

91. The system of claim 88, wherein subjecting said mixture to said reaction conditions comprises applying electromagnetic radiation to said mixture.

92. The system of claim 88, wherein said measuring comprises detecting said signal using a multi-channel detector.

93. The system of claim 88, wherein said signal comprises electromagnetic radiation.

94. The system of claim 93, wherein said electromagnetic radiation comprises a wavelength of electromagnetic radiation.

95. The system of claim 93, wherein said electromagnetic radiation comprises a plurality of wavelengths of electromagnetic radiation.

96. The system of claim 88, wherein said signal is generated by fluorescence emission.

97. The system of claim 88, wherein said signal is generated by chemiluminescence.

98. The system of claim 88, wherein said first range of wavelengths and said second range of wavelengths comprise a same wavelength.

99. The system of claim 88, wherein said first range of wavelengths and said second range of wavelengths do not comprise a same wavelength.

100. The system of claim 88, wherein said detection probe is configured to anneal to said at least one nucleic acid.

101. The system of claim 88, wherein said detection probe is configured to generate said signal upon degradation of a portion of said detection probe.

102. The system of claim 88, wherein said detection probe comprises a fluorophore or dye.

103. The system of claim 88, wherein said mixture further comprises an amplification oligomer.

104. The system of claim 103, wherein said amplification oligomer comprises a sequence complementary to a portion of the sequence of said at least said nucleic acid.

105. The system of claim 88, wherein said reaction conditions comprise conditions for a DNA extension reaction.

106. The system of claim 105, wherein said DNA extension reaction is a polymerase chain reaction (PCR).

107. The system of claim 106, wherein said polymerase chain reaction is a quantitative polymerase chain reaction (qPCR).

108. The system of claim 88, wherein said processing comprises using a mathematical algorithm.

109. The system of claim 108, wherein said mathematical algorithm comprises expectation maximization, nearest neighbor analysis, basic model parameterization or Bayesian estimation.

110. The system of claim 108, wherein said mathematical algorithm comprises a process parameter.

111. The system of claim 110, wherein said process parameter comprises a) a threshold cycle, b) an amplitude, or c) a slope.

112. The system of claim 88, wherein said processing comprises fitting said generated data set to a curve.

113. The system of claim 88, wherein said data set is plotted as a curve.

114. The system of claim 88, wherein said data set is a kinetic signature.

115. The system of claim 88, wherein said reference condition(s) comprises an amplification reaction condition.

116. The system of claim 88, wherein said reference conditions comprise a) a temperature, b) a pH, c) a concentration of said reference nucleic acid, or a combination thereof.

117. The system of claim 88, wherein said reference data set corresponds with a data set generated by amplification of said reference nucleic acid under amplification parameters, wherein said reference data set is indicative of an amplification parameter comprising: a) a primer concentration, b) a polymerase concentration, c) polymerase type, d) a reference nucleic acid concentration, e) a number of thermocycles, f) a rate of thermocycling, g) a thermocycle time length, h) a probe sequence; i) a primer sequence, or combinations thereof.

118. The system of claim 88, wherein said quantifying comprises calculating an absolute quantification.

119. The system of claim 88, wherein said reference data set is generated using predetermined concentrations of said reference nucleic acid.

120. The system of claim 88, wherein said at least one nucleic acid is derived from a biological sample.

121. The system of claim 120, wherein the biological sample is blood or plasma.

122. The system of claim 120, wherein the biological sample is derived from a virus.

123. The system of claim 88, wherein said at least one nucleic acid comprises DNA.

124. The system of claim 123, wherein said at least one nucleic acid comprises genomic DNA.

125. The system of claim 88, wherein said at least one nucleic acid comprises RNA.

126. The system of claim 125, wherein said at least one nucleic acid comprises mRNA.

127. A system for the quantification of at least one nucleic acid in a sample comprising:
a. said sample comprising said at least one nucleic acid;
b. a first detection probe configured to generate a signal when in the presence of said at least one nucleic acid and when subjected to reaction conditions;
c. a detector or plurality of detectors configured to measure (i) an intensity of said signal in a first range of wavelengths and (ii) an intensity of said signal in a second range of wavelengths; and
d. a processor configured to:
i. generate a data set derived from said measured intensities of c); and
ii. process said generated data set by using reference quantification parameters derived from a reference data set(s) to calculate quantification parameters of said generated data set, wherein said reference data set(s) corresponds to a reference condition(s), wherein said reference condition(s) comprises a quantity of reference nucleic acid.

128. The system of claim 127, wherein c) further comprises detectors configured to measure (iii) an intensity of said signal is a third range of wavelengths.

129. The system of claim 127, wherein said reaction conditions comprises applying electromagnetic radiation to said mixture.

130. The system of claim 127, wherein said detector comprises a multi-channel detector.

131. The system of claim 127, wherein said signal comprises electromagnetic radiation.

132. The system of claim 131, wherein said electromagnetic radiation comprises a wavelength of electromagnetic radiation.

133. The system of claim 131, wherein said electromagnetic radiation comprises a plurality of wavelengths of electromagnetic radiation.

134. The system of claim 127, wherein said signal is generated by fluorescence emission.

135. The system of claim 127, wherein said signal is generated by chemiluminescence.

136. The system of claim 127, wherein said first range of wavelengths and said second range of wavelengths comprise a same wavelength.

137. The system of claim 127, wherein said first range of wavelengths and said second range of wavelengths do not comprise a same wavelength.

138. The system of claim 127, wherein said detection probe is configured to anneal to said at least one nucleic acid.

139. The system of claim 127, wherein said detection probe is configured to generate said signal upon degradation of a portion of said detection probe.

140. The system of claim 127, wherein said detection probe comprises a fluorophore or dye.

141. The system of claim 127, wherein said mixture further comprises an amplification oligomer.

142. The system of claim 141, wherein said amplification oligomer comprises a sequence complementary to a portion of the sequence of said at least said nucleic acid.

143. The system of claim 127, wherein said reaction conditions comprise conditions for a DNA extension reaction.

144. The system of claim 143, wherein said DNA extension reaction is a polymerase chain reaction (PCR).

145. The system of claim 144, wherein said polymerase chain reaction is a quantitative polymerase chain reaction (qPCR).

146. The system of claim 127, wherein said processing comprises using a mathematical algorithm.

147. The system of claim 146, wherein said mathematical algorithm comprises expectation maximization, nearest neighbor analysis, basic model parameterization or Bayesian estimation.

148. The system of claim 146, wherein said mathematical algorithm comprises a process parameter.

149. The system of claim 148, wherein said process parameter comprises a) a threshold cycle, b) an amplitude, or c) a slope.

150. The system of claim 127, wherein said processing comprises fitting said generated data set to a curve.

151. The system of claim 127, wherein said data set is plotted as a curve.

152. The system of claim 127, wherein said data set is a kinetic signature.

153. The system of claim 127, wherein said reference condition(s) comprises an amplification reaction condition.

154. The system of claim 127, wherein said reference conditions comprise a) a temperature, b) a pH, c) a concentration of said reference nucleic acid, or a combination thereof.

155. The system of claim 127, wherein said reference data set corresponds with a data set generated by amplification of said reference nucleic acid under amplification parameters, wherein said reference data set is indicative of an amplification parameter comprising: a) a primer concentration, b) a polymerase concentration, c) polymerase type, d) a reference nucleic acid concentration, e) a number of thermocycles, f) a rate of thermocycling, g) a thermocycle time length, h) a probe sequence; i) a primer sequence, or combinations thereof.

156. The system of claim 127, wherein said quantification comprises calculating an absolute quantification.

157. The system of claim 127, wherein said reference data set is generated using predetermined concentrations of said reference nucleic acid.

158. The system of claim 127, wherein said at least one nucleic acid is derived from a biological sample.

159. The system of claim 158, wherein said biological sample is blood or plasma.

160. The system of claim 158, wherein said biological sample is derived from a virus.

161. The system of claim 127, wherein said at least one nucleic acid comprises DNA.

162. The system of claim 161, wherein said DNA comprises genomic DNA.

163. The system of claim 127, wherein said at least one nucleic acid comprises RNA.

164. The system of claim 163, wherein said RNA comprises mRNA.
